(19) United States
(12) Patent Application Publication
(10) Pub. No.: US 2016/0098935 A1
(43) Pub. Date: Apr. 7, 2016

(54) CLINICAL MONITOR EMULATOR FOR CPR FEEDBACK

(71) Applicant: THE JOHNS HOPKINS UNIVERSITY, Baltimore, MD (US)

(72) Inventors: Jordan Duval-Arnould, Baltimore, MD (US); Julie Campbell, Westerly, RI (US); Elizabeth Hunt, Stevensville, MD (US)

(21) Appl. No.: 14/868,963

(22) Filed: Sep. 29, 2015

Related U.S. Application Data
(60) Provisional application No. 62/059,187, filed on Oct. 3, 2014.

(51) Int. Cl. G09B 5/06; G06F 3/16; G06F 3/14; H04N 5/225

(52) U.S. Cl. CPC G09B 5/06; H04N 5/225; G06F 3/16; G06F 3/1454

(57) ABSTRACT

An embodiment in accordance with the present invention is directed to an electronic training tool having one or more sensor components. The sensor components include a single depth camera and accelerometer or force sensor. The training tool also includes a software component for signal processing of sensor data and analysis of user performance. The user is given feedback based on quality of chest compressions, which is determined using data related to depth of compressions, rate of compressions, and recoil of compressions. Feedback is presented to the user and/or the trainer of the user via a user interface governed by a software program that can be run on a computing device such as a pc, laptop, tablet, smartphone, etc. The user interface imitates the user interface of a clinical monitor/defibrillator, specifically a defibrillator with a built-in QCPR feedback functionality.

### CROSS REFERENCE TO RELATED APPLICATIONS

This application claims the benefit of U.S. Provisional Patent Application No. 62/059,187 filed Oct. 3, 2014, which is incorporated by reference herein, in its entirety.

### FIELD OF THE INVENTION

The present invention relates generally to medical devices. More particularly, the present invention relates to a clinical monitor emulator for providing feedback on CPR technique.

### BACKGROUND OF THE INVENTION

The American Heart Association's (AHA) Guidelines for Cardiopulmonary Resuscitation and Emergency Cardiovascular Care state that quality cardiopulmonary resuscitation (CPR) is that in which the rescuers push hard, push fast, allow for complete chest recoil between compressions, and minimize interruptions in compressions for all victims. In addition, the AHA recommends that the rate and tidal volume of ventilations should be decreased to reduce inadvertent hyperventilation and reduce intrathoracic pressures, two things shown to worsen chances of survival for patients suffering from sudden cardiac arrest (SCA). Studies also indicate that the application of quality CPR is a major variable in influencing the likelihood of survival.

It is therefore important to provide training in effective, quality CPR. Current training in quality CPR includes use of a clinical defibrillator with a monitor. However, use of such a clinical defibrillator in training reduces the number of providers that can train at once. In CPR and basic life support (BLS) classes, clinical defibrillators with monitors are used to demonstrate to trainees in a lecture-style. The trainees receive limited hands-on training because allocation of clinical defibrillators and monitors for training purposes often is not feasible. As a result, the trainees do not receive feedback on the quality of their application of the CPR method.

Accordingly, there is a need in the art for a system and method to facilitate quality CPR training with a monitor and feedback on the quality of the CPR applied.

### SUMMARY OF THE INVENTION

The foregoing needs are met, to a great extent, by the present invention which provides a system for training a user in CPR including a sensor component configured to detect compressions in a CPR training event and configured to output data related to the compressions in the CPR training event. The system also includes a non-transitory computer readable medium. The non-transitory computer readable medium is programmed with steps including receiving the data from the sensor component. The steps also include identifying user motion representative of chest compressions and chest decompressions during the CPR training event and calculating metrics related to the chest compressions and the chest decompressions. Additionally, the steps include comparing the metrics to preset goals and displaying the metrics and a comparison of the metrics to the goals to the user.

In accordance with another aspect of the present invention the sensor can take the form of at least one selected from a group consisting of single depth camera, accelerometer, and force sensor. The accelerometer and force sensor are incorporated into a clinical defibrillator or can be a standalone component designed specifically for the system. The metrics can take the form of at least one selected from a group consisting of chest compression rate, chest decompression rate, depth of compressions, and recoil of compressions. The system includes a user display. The non-transitory computer readable medium can be loaded onto a computing device, which can take the form of a laptop computer, a personal computer, a tablet, a phablet, or a smartphone.

In accordance with another aspect of the present invention the non-transitory computer readable medium is further programmed for preparing a score sheet for the user. The score sheet further includes graphs indicating chest compression depth and showing chest compression rate. The non-transitory computer readable medium is further programmed for providing visual and auditory feedback to the user during the CPR training event.

In accordance with still another an aspect of the present a method for training a user in CPR includes detecting compressions in a CPR training event with a sensor component configured to output data related to the compressions in the CPR training event. The method includes receiving the data from the sensor component and identifying user motion representative of chest compressions and chest decompressions during the CPR training event. The method includes calculating metrics related to the chest compressions and the chest decompressions and comparing the metrics to preset goals. Additionally, the method includes displaying the metrics and a comparison of the metrics to the goals to the user, such that the user can apply the feedback for improving the chest compressions and decompressions in real time.

In accordance with yet another aspect of the present invention, the method includes using a sensor taking a form of at least one selected from a group consisting of single depth camera, accelerometer, and force sensor. The method includes incorporating the accelerometer and force sensor into a clinical defibrillator. Collecting the metrics can take the form of at least one selected from a group of chest compression rate, chest decompression rate, depth of compressions, and recoil of compressions. A user display is available for inputting information. The method is executed with a non-transitory computer readable medium. The non-transitory computer readable medium is loaded onto a computing device comprising one chosen from a group consisting of a laptop computer, a personal computer, a tablet, a phablet, and a smartphone. A score sheet can be prepared for the user. The method includes indicating chest compression depth and showing chest compression rate on the score sheet. Additionally, the method includes providing visual and auditory feedback to the user during the CPR training event.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a schematic diagram of the electronic training tool, according to an embodiment of the present invention.

FIGS. 2A and 2B and 3A and 3B illustrate exemplary images of user feedback screens, according to an embodiment of the present invention.

FIGS. 4A and 4B illustrate configurations of the score reporting form and exported as html and presented in a web browser, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention is directed to an electronic training tool having one or more sensor components. The sensor component can include one or more of a single depth camera, accelerometer, or force sensor. The training tool also includes a software component for signal processing of sensor data and analysis of user performance. The user is given feedback based on quality of chest compressions, which is determined using data related to depth of compressions, rate of compressions, and recoil of compressions. Feedback is presented to the user and/or the trainer of the user via a user interface governed by a software program that can be run on a computing device such as a pc, laptop, tablet, smartphone, etc. The user interface imitates the user interface of a clinical monitor/defibrillator, specifically a defibrillator with a built-in QCPR feedback functionality.

The present invention is designed to supplement realistic, hands-on quality CPR (QCPR) training to a user by emulating the clinical monitor/defibrillator screen on a screen of a computing device. The user interface can be adapted to match different monitor modes, and the real time feedback visually and audibly matches the feedback provided by a real clinical monitor or defibrillator screen. The emulator can be easily modified and updated, allowing for effective assessment of clinical monitor/defibrillator under interfaces. Using the modifiable emulator medical device manufacturers and researchers will be better able to study interactions of providers and the interface and characterize any resulting issues.

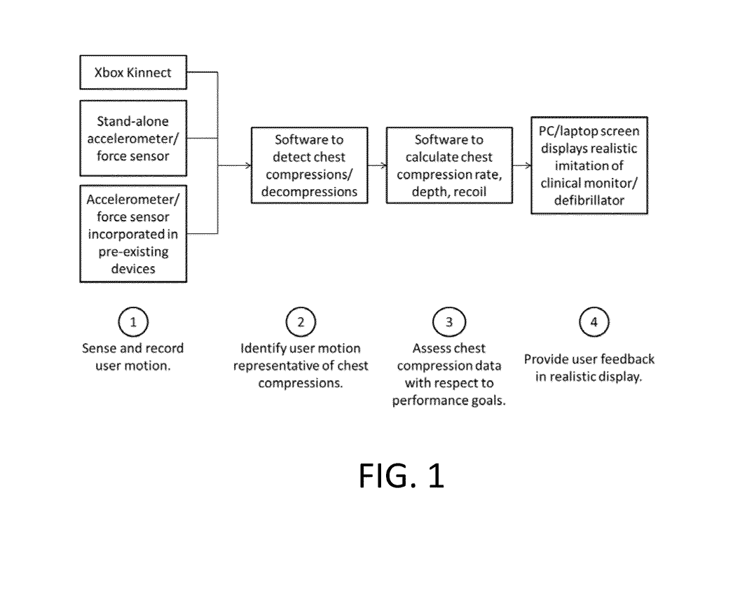

FIG. 1 illustrates a schematic diagram of the electronic training tool, according to an embodiment of the present invention. FIG. 1 shows an exemplary workflow of the invention, which is formatted to provide QCPR feedback in a realistic format, matching the clinical monitor/defibrillator. Step 1 includes sensing and recording user motion. The sensing and recording of user motion can be done in a number of ways for instance with a single depth camera, an accelerometer or a force sensor. The single depth camera can take the form of an Xbox Kinnect® or any other suitable depth camera. The accelerometer or force sensor can be a standalone component that is made specifically for the invention. Alternately, the accelerometer or force sensor can take the form of hardware from a clinical monitor/defibrillator device. An exemplary clinical monitor/defibrillator device has a hardware piece, which includes clinical defibrillator pads with integrated accelerometer (such as ZOLL OneStep Complete Pads) and an analog-to-digital converter. The analog-to-digital converter and associated hardware completes minimal smoothing of the raw accelerometer data and employs serial communication to transmit data to the computer on which the emulator software is loaded/running The emulator software filters digital accelerometer data and calculates the real-time position of the accelerometer; compression depth, rate, and recoil are evaluated from calculated position data.

Further with respect to FIG. 1, the data from the single depth camera and the accelerometer is transmitted to a computing device running the emulator software. In step 2, the software processes the data to detect chest compressions/decompressions, and generally identifies user motion representative of chest compressions. In step 3, the software assesses chest compression data with respect to personal performance goals or preset performance goals. The software calculates chest compression rate, depth and recoil. Additionally, in step 4, the software provides user feedback in a realistic display. The display can provide a comparison of the user's performance to the goals and also offer suggestions on how to improve performance.

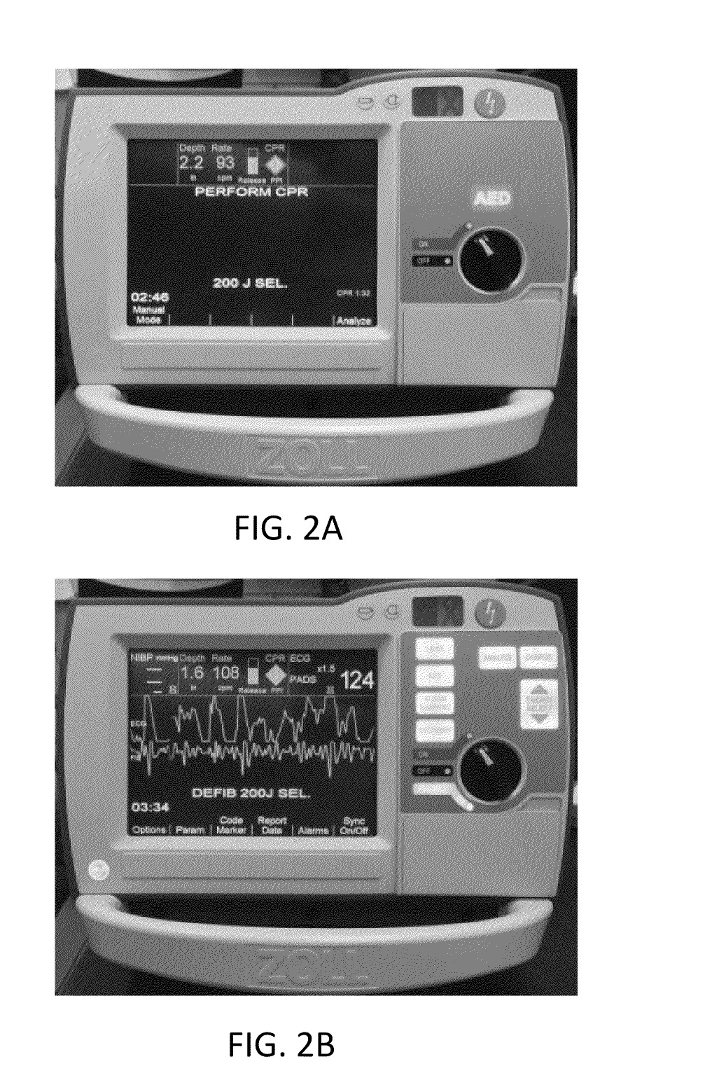

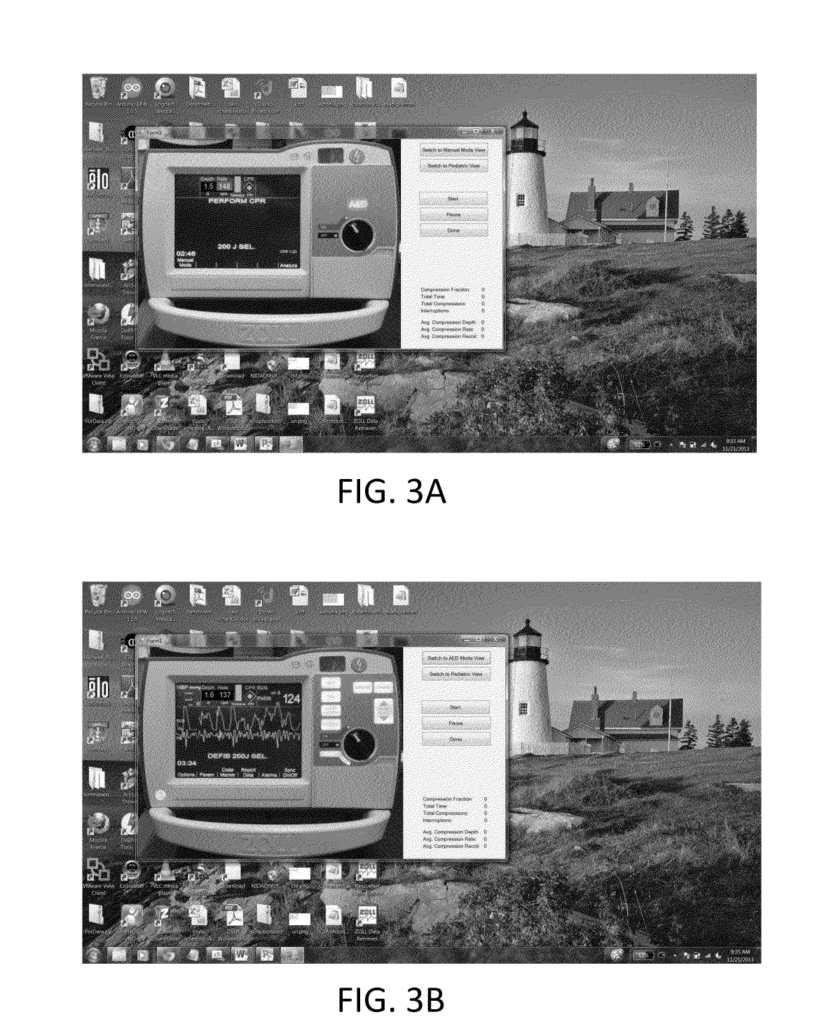

FIGS. 2A and 2B and 3A and 3B illustrate exemplary images of user feedback screens, according to an embodiment of the present invention. The emulator screen imitates the various modes of the ZOLL R Series defibrillator, as shown in FIGS. 2A, 2B, 3A, and 3B. The QCPR feedback located in the top, center box of the screen is updated in real-time based on user CPR performance. The emulator imitates this real-time functionality through visual and audio cues that match those of the defibrillator. The user feedback is constantly updated on the user feedback screens based on the user's performance of the CPR. This feedback allows the user to instantly improve performance. The user's changes to performance are then reflected in real time on the feedback screens. This also allows the user to experiment with the results of different depths of compression.

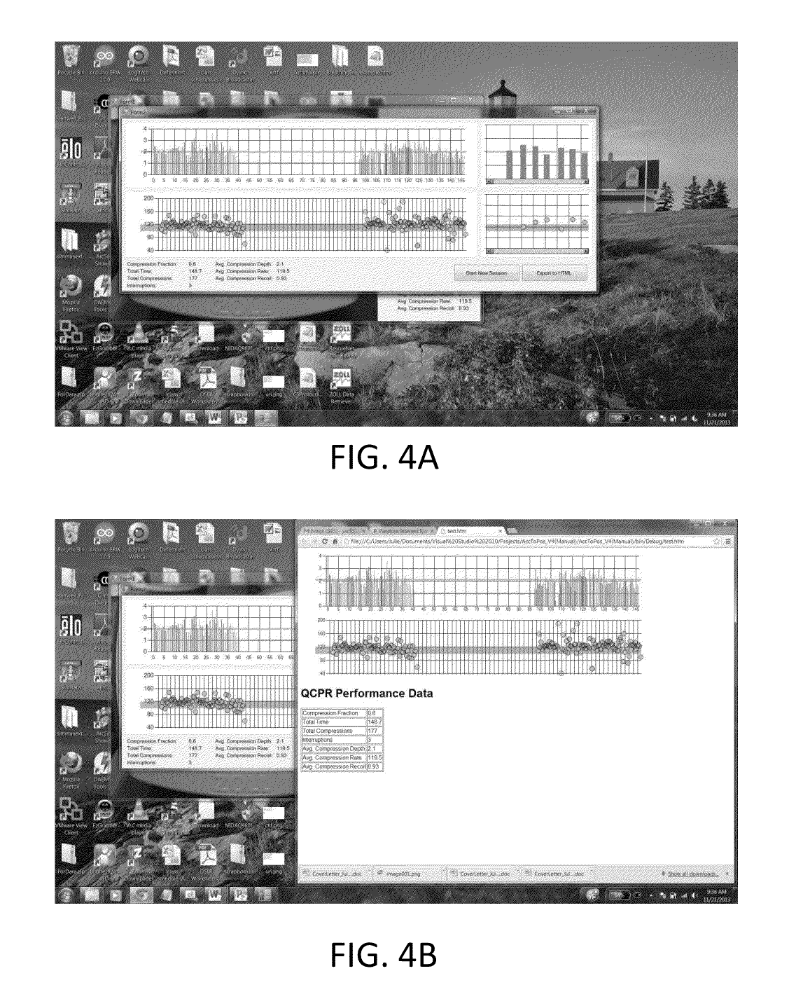

The emulator also will provide a score sheet at the end of training The current format of this score sheet includes 2 plots, one indicating compression depth by bar height and the second showing the compression rate charted on the Y axis as circles with the depth indicated by the extent of circle fullness. This score report can be exported allow for printing and saving. Current configurations of the score reporting form and exported as html and presented in a web browser are illustrated in FIGS. 4A and 4B.

The emulator software can also include built in score-reporting functionalities. Not only do users receive real-time feedback while performing CPR, as they would clinically, but also, once the trainee has finished a training session, a printable score sheet with both visual and numeric feedback is provided. Although the present invention is outfitted to provide QCPR feedback in a realistic format, additional functionality can also be added. For instance, the present invention could include training of device navigation (i.e. button/key presses on device), waveform recognition (ECG, ETCO₂), demonstration of device features, etc.

The emulator can also be configured such that the user continues training until the personal or selected preset performance goals are met. Once the performance goals are met, the emulator can be configured to end training and provide the user with a score report and a summary of the training It is also possible that if the user does not meet the goals within a predetermined period of time, the emulator ends the training and provides the user with the score report, a summary of the training, a list of the goals that were met and those that were unmet, and areas for improvement of technique in order to meet the goals.

It should be noted that the methods described herein can be executed with a program(s) fixed on one or more non-transitory computer readable medium. The non-transitory computer readable medium can be loaded onto a computing device, server, smartphone, tablet, phablet, or any other suitable device known to or conceivable by one of skill in the art. It should also be noted that herein the steps of the method described can be carried out using a computer, non-transitory computer readable medium, or alternately a computing device, microprocessor, or other computer type device independent of or incorporated with a signal collection device.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

The many features and advantages of the invention are apparent from the detailed specification, and thus, it is intended by the appended claims to cover all such features and advantages of the invention, which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A system for training a user in CPR comprising:
a sensor component configured to detect compressions in a CPR training event and configured to output data related to the compressions in the CPR training event;
a non-transitory computer readable medium programmed with steps comprising:
receiving the data from the sensor component;
identifying user motion representative of chest compressions and chest decompressions during the CPR training event;
calculating metrics related to the chest compressions and the chest decompressions;
comparing the metrics to preset goals; and
displaying the metrics and a comparison of the metrics to the goals to the user, such that the user can apply the feedback for improving the chest compressions and decompressions in real time.

2. The system of claim 1 further comprising the sensor taking a form of at least one selected from a group consisting of single depth camera, accelerometer, and force sensor.

3. The system of claim 2 wherein the accelerometer and force sensor are incorporated into a clinical defibrillator.

4. The system of claim 1 further comprising the metrics taking the form of at least one selected from a group consisting of chest compression rate, chest decompression rate, depth of compressions, and recoil of compressions.

5. The system of claim 1 further comprising a user display.

6. The system of claim 1 further comprising the non-transitory computer readable medium being loaded onto a computing device.

7. The system of claim 6 further comprising the computing device comprising one chosen from a group consisting of a laptop computer, a personal computer, a tablet, a phablet, and a smartphone.

8. The system of claim 1 wherein the non-transitory computer readable medium is further programmed for preparing a score sheet for the user.

9. The system of claim 8 wherein the score sheet further comprises indicating chest compression depth and showing chest compression rate.

10. The system of claim 1 wherein the non-transitory computer readable medium is further programmed for providing visual and auditory feedback to the user during the CPR training event.

11. A method for training a user in CPR comprising:
detecting compressions in a CPR training event with a sensor component configured to output data related to the compressions in the CPR training event;
receiving the data from the sensor component;
identifying user motion representative of chest compressions and chest decompressions during the CPR training event;
calculating metrics related to the chest compressions and the chest decompressions;
comparing the metrics to preset goals; and
displaying the metrics and a comparison of the metrics to the goals to the user, such that the user can apply the feedback for improving the chest compressions and decompressions in real time.

12. The method of claim 11 further comprising using a sensor taking a form of at least one selected from a group consisting of single depth camera, accelerometer, and force sensor.

13. The method of claim 12 further comprising incorporating the accelerometer and force sensor into a clinical defibrillator.

14. The method of claim 11 further comprising collecting the metrics taking the form of at least one selected from a group consisting of chest compression rate, chest decompression rate, depth of compressions, and recoil of compressions.

15. The method of claim 11 further comprising inputting information with a user display.

16. The method of claim 11 further comprising executing the method with a non-transitory computer readable medium.

17. The method of claim 16 further comprising loading the non-transitory computer readable medium on a computing device comprising one chosen from a group consisting of a laptop computer, a personal computer, a tablet, a phablet, and a smartphone.

18. The method of claim 11 further comprising preparing a score sheet for the user.

19. The method of claim 18 further comprising indicating chest compression depth and showing chest compression rate on the score sheet.

20. The method of claim 11 further comprising providing visual and auditory feedback to the user during the CPR training event.
